(19) United States
(12) Patent Application Publication
(10) Pub. No.: US 2007/0248695 A1
(43) Pub. Date: Oct. 25, 2007

(54) LANDSCAPING, PLANT POTTING, AND HORTICULTURE METHODS ACCORDING TO THE TRADITIONAL ORIENTAL OR KOREAN MEDICINE

(76) Inventor: Won-Gon KIM, Gyungsangnam-Do (KR)

Correspondence Address: LADAS & PARRY LLP, 224 SOUTH MICHIGAN AVENUE, SUITE 1600, CHICAGO, IL, 60604, US

(21) Appl. No.: 11/742,928

(22) Filed: May 1, 2007

Related U.S. Application Data
(62) Division of application No. 10/478,429, filed on Nov. 21, 2003, now abandoned.

(30) Foreign Application Priority Data
May 23, 2001 (KR) 10-2001-0028438

(51) Int. Cl. A61K 36/258; A01B 79/02; A61K 36/00; A61K 36/48; A61K 36/54; A61K 36/28; A61K 36/13

(52) U.S. Cl. 424/728; 424/725; 424/739; 424/757; 424/764; 424/770; 47/58.1 R

(57) ABSTRACT

Plants that are substantially utilized as the medicinal ingredients in a selected single prescription of the traditional Oriental and Korean medicine are planted together in close proximity to each other. The plants provide therapeutic effects to the body of a person breathing the fragrance emitted by the plants and observing the plants. The plants are selected in accordance with the prescription of Sagunja-tang (a ginseng; a Atractylodes japonica koidz; a Glycyrrhiza uralensis Fisch; and a Pinus densiflora) or Samul-tang (an Angelica gigas Nakia; a Cnidium oficinale; a Paeonia lactiflora Pall; and a Rhemanniae glutinosa Libosch) or Palmul-tang (the eight plants selected according to Sagunja-tang and Samul-tang) or Shipjundaebo-tang (the eight plants according to Palmul-tang plus an Astrualus membranaceus Bunge var and a Cinnamon). These plants can be planted in a portable medium including at least a flowerpot.

### BACKGROUND OF INVENTION

1. Field of Invention

This application relates generally to landscaping, plant potting, and/or horticulture methods and more particularly to landscaping, plant potting, and/or horticulture methods utilizing various plants, trees, flowers, herbs, etc. that are also the medicinal ingredients of the traditional Oriental or Korean medicine.

2. Description of Prior Art

The traditional Oriental or Korean medicine (hereinafter collectively referred to as “the Eastern medicine” or “the Oriental medicine”) has been in practice for thousands of years in Korea and in other Eastern countries such as China. The history and the philosophy of the Oriental medicine is generally described hereinbelow as the background for the embodiments of the present invention.

Generally, the landscaping, gardening, plant potting, or other types of horticulture in general (hereinafter collectively referred to as “the landscape” or “the landscaping”) involves placing, arranging, or planting the natural and/or the artificial elements on a soiled surface so as to provide a beautiful, useful, and healthy contributions to the human living environments. To achieve this, it is not only important to utilize the creative and technical skills of those who are creating the landscape, but also important to utilize the knowledge that is based on the science, the culture, and the emotional desires of the heart, soul, and mind of the people who desire the landscape.

The landscaping techniques around the world have changed over time. They have generally related to the general psyche of the people of their country, their religion, their history, the climate and natural features of the country, their customs and habits, and the landscaping owner's interests, etc. The landscaping techniques can be broadly categorized as: the natural landscaping technique, the artificial landscaping technique, and the sociological landscaping technique. Further, the landscaping techniques can be geographically categorized as the Western technique and the Eastern technique.

The landscaping can be done for indoors or outdoors. The indoor landscaping is considered to bring the nature into the boundaries of one's close surroundings such as his home or his office, for example, by cultivating a garden in a yard inside the house or, if no outdoor yards are available, by planting flowers and plants in pots inside the offices, living rooms, etc. One trend these days for the indoor landscaping (as it is also true for the outdoor landscaping) is to focus on promoting the better health of the people by planting plants such as herbal plants, cacti, etc. that, among others, provide pleasant aroma. Thus, the various benefits of nature, such as the fresh air for the healthy environment, along with the aesthetic beauty, can be brought indoors.

In considering the interrelationship between the plants and human beings, they have been understood by the Eastern medicine as being very closely interrelated. In many Eastern countries including Korea and China, this interrelationship between the plants and human beings has been studied and researched for a long period of time, and has been evolved into an Eastern or Oriental medical science.

For example, “Shikmul Bohncho” (which is a medical book written during the mid-Chosun Dynasty of Korea) records how various human foods can be utilized for curing various human illnesses. This book was introduced by Yo Moon Chung in 1526 in the Myung Dynasty of China and was republished in Korea thereafter in around 1521-1567. Shikmul Bohncho in Volumes I & II divides the foods into 8 different groups (water, grain, vegetable, fruit, poultry, animal, fish, and taste), and they are further divided into 387 articles. Each of these articles describes the plants' (or the foods') tastes, general characteristics, positive effects and benefits, harmful elements, etc. Along with these articles, Shikmul Bohncho also includes a collection of treatises on various kinds of plants.

“Shinnong Bohncho Kyung” (a medical book about the vegetation, animals, and natural resources used in the Chinese medical science), was published by Do Hong Kyung during the Chinese Yang Dynasty. Shinnong Bohncho Kyung classifies many ingredients of the Oriental medicine into superior, middle, and inferior categories and describes the effects of each medicine. Shinnong Bohncho Kyung describes that there are generally 120 superior medicines, 120 middle medicines, and 125 inferior medicines.

The Oriental Medicine has been developed through these publications and other processes, and it was generally summed up in “Dong-i Bogam” and “I-Bang Yoochi” published in Korea also during the Chosun Dynasty.

The medicinal ingredients that are often prescribed by the Oriental medicine are typically a mixture of various medicinal plants and herbs. A prescription for a mixture of herbs is based on the careful consideration of each herb's effect on pathology, physiology, and natural medicinal effects. For example, an Oriental medicine prescription may prescribe a mixture of the primary herbs, the secondary herbs, the herbs that would balance the effectiveness of the medicine, and the herbs that would lead to the efficacy of a remedy to where an illness resides, and many other herbs that are all precisely selected according to its known medicinal effects. The medicinal effects of the natural herbs and other medicinal ingredients utilized in the Oriental medicine is known as the “kimi” or the inner workings, and this theory of “kimi” is considered to be the basic doctrine of the Oriental medicine.

In this regard, the Oriental medicine has been considered by the East and the West as the science and philosophy. The Oriental medicine has matured to its present form practiced in Korea and China and in other parts of the world based on the continuing analysis of the natural herbs or other components of medicine and the effects of the medicine through the results and experiences of the clinical trials.

However, although the Oriental medicine is extensively being used as a tool for treating patients, the Oriental medicine, unfortunately, has not been put into practical use such as in landscaping.

Therefore, the present invention is directed to applying the Oriental medicine into practical use as in landscaping. Of the Oriental medicinal prescriptions including the acupuncture, the extraction of pus or blood, the herbal medicine, the pills, etc., the prescriptions of the herbal medicines and pills are generally applied and utilized in the landscaping techniques of the present invention as they are further described in detail in various embodiments hereinbelow. The natural disposition of this invention utilizing the well-known prescriptions of the Oriental medicine is also based on “Dong-I Bogam,” “I-Bang Yoochi,” “Dong-I Soose Bowon,” and other known Oriental medicine publications.

### SUMMARY OF THE INVENTION

Against this backdrop, embodiments of the present invention have been developed.

First, the methods of naming landscapes formed according to an oriental medicinal prescription is described hereinbelow. The name of the landscape formed by using at least one of the plants that are the ingredients of Sagunja-tang (including ginseng, Atractylodes japonia koidz, Glycyrrhiza uralensis Fisch, and Pinus densiflor) includes the Sagunja-tang in its landscape name.

The name of the landscape formed by using at least one of the plants that are the ingredients of Samul-tang (Angelica gigas Nakai, Cnidium oficinale, Paeonia lactiflora Pall, and Rehmannia glutinosa Libosch) includes the Samul-tang in its landscape name, The name of the landscape formed by using the plants that are the ingredients of Palmul-tang (ginseng, Atractylodis Rhizoma, Glycyrrhizae Radix, Poria cocos, Angelicae gigantis radix, Cnidium rhizoma, Paeoniae radix and Rehmanniae radix preparata) includes the Palmul-tang in the landscape name.

The name of the landscape formed by using the plants that are the ingredients of Shipjundaebo-tang (ginseng, Atractylodis Rhizoma, Glycyrrhizae Radix, Poria cocos, Angelicae gigantis radix, Cnidium rhizoma, Paeoniae radix, Rehmanniae radix preparata, Astrugalus membranaceus Bunge var and Cinnamon) includes the Shipjundaebo-tang in the landscape name.

Likewise, the name of the plant potting formed by using the plants that the ingredients of Sagunja-tang (ginseng, Atractylodes japonia koidz, Glycyrrhiza uralensis Fischand Pinus densiflor) includes the Sagunja-tang in the plant potting name,

The name of the plant potting formed by using the plants that the ingredients of Samul-tang (Angelica gigas Nakai, Cnidium oficinale, Paeonia lactiflora Pall, and Rehmannia glutinosa Libosch) includes the Samul-tang in the plant potting name,

The name of the plant potting formed by using the plants that are the ingredients of Palmul-tang (ginseng, Atractylodis Rhizoma, Glycyrrhizae Radix, Poria cocos, Angelicae gigantis radix, Cnidium rhizoma, Paeoniae radix and Rehmanniae radix preparata) includes the Palmul-tang in the plant potting name,

The name of the plant potting formed by using the plants that are the ingredients of Shipjundaebo-tang (ginseng, Atractylodis Rhizoma, Glycyrrhizae Radix, Poria cocos, Angelicae gigantis radix, Cnidium rhizoma, Paeoniae radix, Rehmanniae radix preparata, Astrugalus membranaceus Bunge var and Cinnamon) includes the Shipjundaebo-tang in the plant potting name.

Second, the method of landscaping according to various embodiments of the present invention is described hereinbelow. A landscape is formed by using the plurality of plants that are named as the ingredients of at least one of the oriental medicine prescriptions. The oriental medicine prescriptions comprise: a Sagunja-tang, a Samul-tang, a Palmul-tang, a Shipjundaebo-tang.

The Sagunja-tang comprises ginseng, Atractylodes japonia koidz, Glycyrrhiza uralensis Fisch, and Pinus densiflor. The Samul-tang comprises Angelica gigas Nakai, Cnidium oficinale, Paeonia lactiflora Pall, and Rehmannia glutinosa Libosch. The Palmul-tang comprises ginseng, Atractylodis Rhizoma, Glycyrrhizae Radix, Poria cocos, Angelicae gigantis radix, Cnidium rhizoma, Paeoniae radix, and Rehmanniae radix preparata. The Shipjundaebo-tang comprises ginseng, Atractylodis Rhizoma, Glycyrrhizae Radix, Poria cocos, Angelicae gigantis radix, Cnidium rhizoma, Paeoniae radix, Rehmanniae radix preparata, Astrugalus membranaceus Bunge var, and Cinnamon.

Similarly, a plant potting, which may be movable, is formed by using the plurality of plants that are named as the ingredients of at least one of the oriental medicine prescriptions comprising: a Sagunja-tang, a Samul-tang, a Palmul-tang, and a Shipjundaebo-tang, as the ingredients of these prescriptions are described above.

Further, a collection of two or more plants that are named as the ingredients in one of oriental medicine prescriptions is cultivated. The oriental medicine prescriptions comprise: a Sagunja-tang, a Samul-tang, a Palmul-tang, and a Shipjundaebo-tang, as the ingredients of these prescriptions are described above.

Finally, plants, which are substantially utilized as the medicinal ingredients in a selected single prescription of the traditional Oriental and Korean medicine, planted together in close proximity to each other. The plants provide therapeutic effects to the body of a person breathing the fragrance emitted by the plants and observing the plants.

The plants are selected from the group of plants in accordance with the prescription of Sagunja-tang, including: a ginseng; a Atractylodes japonica koidz; a Glycyrrhiza uralensis Fisch; and a Pinus densiflora. These plants may be planted in a portable medium including at least a flowerpot.

The plants are selected from the group of plants in accordance with the prescription of Samul-tang including: an Angelica gigas Nakia; a Cnidium oficinale; a Paeonia lactiflora Pall; and a Rhemanniae glutinosa Libosch. These plants may be planted in a portable medium including at least a flowerpot.

The plants are selected from the group of plants in accordance with the prescription of Palmul-tang, including: a ginseng; a Atractylodes japonica koidz; a Glycyrrhiza uralensis Fisch; a Pinus densiflora; an Angelica gigas Nakia; a Cnidium oficinale; a Paeonia lactiflora Pall; and a Rhemanniae glutinosa Libosch. These plants may be planted in a portable medium including at least a flowerpot.

The plants are selected from the group of plants in accordance with the prescription of Shipjundaebo-tang, inclduing: a ginseng; a Atractylodes japonica koidz; a Glycyrrhiza uralensis Fisch; a Pinus densiflora; an Angelica gigas Nakia; a Cnidium oficinale; a Paeonia lactiflora Pall; a Rhemanniae glutinosa Libosch; an Astrualus membranaceus Bunge var; and a Cinnamon. These plants may be planted in a portable medium including at least a flowerpot.

These and various other features as well as advantages which characterize the present invention will be apparent from a reading of the following detailed description and a review of the associated drawings, if any.

### BRIEF DESCRIPTION OF THE DRAWINGS

No drawings are submitted herewith.

### DETAILED DESCRIPTION

Because the embodiments of the present invention are directed to landscaping, gardening, flower planting, plant potting, or other types of horticulture in general (hereinafter collectively referred to as “the landscaping” or “the landscape”) architecture, techniques, and methods that are based on the Oriental medicine and its medicinal prescriptions, the emphasis is put on the healing effects of the specific herbs, plants, or other landscaping components on specific human illnesses, more so than the emphasis put on by the conventional landscaping methods to provide visually pleasing landscape environment. There is simplicity since the landscape architecture of the present invention is developed to combine mainly the herbs and plants of the Oriental medicine based on the medicinal elements and effects.

The solutions to various problems provided by the present invention are:

1. In providing the landscape according to the Oriental medicine that often prescribes herbs and plants/trees that grow in different growing environments, the present invention provides the landscaping techniques that would overcome the different growing environments. For example, if a prescription according to the Oriental medicine may prescribe two herbs that grow in two different environments with a big temperature difference, such as Cnidium officinale (growing in a cold environment) and a cinnamon tree (growing in a hot region), either one or both will not be planted in ground but can be developed in interior herbal pots for landscaping.

2. In providing the landscape according to the Oriental medicine prescription that prescribes non-medicinal plants that are rather difficult to cultivate on soil, such as medicinal animals, mushrooms, nodes of roots, etc., they can be replaced with substitute plants and materials, which may have the related or similar medicinal effects. For example, the antlers of a young deer may be substituted with a model of a deer or may be omitted. Likewise, a node of a pine tree's root, which may be one of the prescribed elements in the Oriental medicine, can be omitted or can be substituted with a pine tree as the landscaping material.

3. The landscaping techniques according to the present invention distinguish itself from the conventional landscaping techniques that emphasize mainly the visual effect and beauty. The present invention utilizes the Oriental medicine prescriptions for the landscaping and plant potting, thus the present invention creates new landscape architecture formations and plant potting formations based on the classification of plants and their effects and benefits according to the Oriental medicine.

4. Various materials will be developed in the present invention in landscaping utilizing herbal medicinal elements of the oriental medicine.

Generally, the Western medicine is considered to be based on natural sciences and is considered to be analytical and emphasizing the study of human body at the level of cellular formation and structure. Thus, the Western medicine is regarded as being very effective in defending and eliminating the external factors of diseases, which may threaten the life of a human being. In comparison to the Western medicine, the Oriental medicine is considered to be taking a holistic approach and effective in promoting the health and the healing effects internally.

The herbs, plants, flowers and other materials used in the Oriental medicine (hereinafter collectively referred to as “the medicinal ingredients”) have, among others, the quality of fragrance that is considered to provide healing effects. The present invention is directed to combining the elements and effects of the medicinal ingredients prescribed in the Oriental medicine to develop the new techniques and formations for landscaping, gardening, the landscape architecture, plant potting, flower planting, etc. that include the plants, herbs, flowers, etc. (or effective substitutes where appropriate) according to the prescriptions of the Oriental medicine for curing specific illnesses.

Therefore, various landscaping methods that are based on the medicinal ingredients of the prescriptions the Oriental medicine are described in various embodiments of the present invention described herein. The present invention relies on the Oriental medicine publications such as “Dong-I Bogam,” “I-Bang Yoochi” and “Dong-I Soose Bowon” as references for the prescriptions, and the materials used for landscaping in the present application are primarily medicinal plants and herbs.

The following are the examples of the Oriental medicine prescriptions (Sagunja-tang, Samul-tang, Palmul-tang, and Shipjundaebo-tang) and the detailed description of the landscape architecture and flower planting formations of the present invention. Although the embodiments of the present invention are described with respect to the four Oriental medicine prescriptions, namely Sagunja-tang, Samul-tang, Palmul-tang, and Shipjundaebo-tang, it should be readily understood that other Oriental medicine prescriptions may also be utilized without departing the scope and spirit of the present invention.

### A. Sagunja-tang

Oriental Medicine Prescription: Sagunia-tang

1. Classification: prescription for men.
2. Main ingredients: ginseng, Atractylodis Rhizoma, Glycyrrhizae Radix, Poria cocos.
3. Symptoms treated by the prescription: Sagunja-tang is known as one of the superior prescription of the Oriental medicine, which is considered to parallel the Samul-tang, which is prescribed for women in the medicinal effects. The general symptoms treated by the Sagunja-tang include chronic fatigue, weary back and legs, weak energy and tiredness often caused by stress and/or the lack of exercise. Sagunja-tang strengthens weak body energy and restores the body to be full of energy.
4. General Effects:

a. The ginseng activates the capacity of muscle, saponin and strengthens the reflective ability of the body, thus strengthening the body energy. The ginseng is known to reinforce the body immune system. The Poria cocos' nutritional factors of amino acids, protein, lecithin, Kalium, etc. supply nutritions to body cells. Atractylodis Rhizoma supplies vitamin A & D

b. The ginseng and Atractylodis Rhizoma contains elements that strengthen digestive system and improve digestion and absorption.

c. Atractylodis Rhizoma and Poria cocos promote the discharge of accumulated stomach waste.

The “Sagunia-tang” Landscape Architecture

This formation of landscape architecture utilizes the Sagunja-tang prescription of the Oriental medicine and the prescribed herbs' effects and benefits classified according to the symptoms. The name, “Sagunja-tang,” is also used in the name of the landscape architecture to create familiarity.
1. Main landscaping materials: ginseng, Atractylodes japonia koidz, Glycyrrhiza uralensis Fisch, and Pinus densiflora
2. Characteristics of each main landscaping material:

a. The ginseng contains various elements including saponin, an element of glycoside, nitrogen chemical combinations of protein, amino acids, nucleic acid, alkaloid, fat solubles of fatty acid, essential oil, polyethylene, phenol, phytosterol, terpene, various saccharide of monosaccharide, oligosacchride, polysaccharide, pectin, various types of vitamins, inorganic substances and etc.

b. Atractylodes japonia koidzcontains inulin, aquatic calcium, raphides, vitamin D as well as the essential oils of Hinesol, Atractylone, Atractulenolide I, II, III, beta-Eudesmol.

c. Glycyrrhiza uralensis Fisch contains glycerine, flavonoid, glcerol acid, saponin, coumarin essential oil, starch, phytosterol, tannin, enzymes, etc, and the primary ingredient, glycerine's sweetness is 50 times more than saccharose.

d. Pinus densiflora contains terpene on its pine needles, phenol chemical combinations, tannin, etc.

3. The medicinal effects of each landscaping material:

a. The ginseng strengthens the overall body health and the stomach (the digestive system); nourishes the blood; prevents stagnant appetite, menopausal difficulties, and weariness or helps to recuperate therefrom; protects the body from the rheumatism, the diabetes, liver problems, sexual impotence, the lack of sperm, and aging.

b. Atractylodes japonia koidz improves urination and perspiration to cure the impure water replacement ability of the body; helps to heal general aches, gastritis, and swelling of the body; and is also used as a blood pressure stimulant.

c. Glycyrrhiza uralensis Fisch manages the digestion, the circulation, and the otorhinolaryngologic disorders. It is also used as a neutralizer and/or an antidote.

d. Pinus densiflora helps the body to relax as if the body is resting in a forest. Its scent helps to manage and improve the blood circulation, the pulse rate, and the blood pressure of the body. (The primary element of the pine needles, terpene, is a scent with volatility, which stimulates the brain to excite or relax once it comes in contact with the skin and the mucous membrane. Thus, it is used in an aromatherapy.)

4. The characteristics of the Sagunja-tang landscape architecture:

As an herbal medicine, Sagunja-tang is a type of restoration medicine to revive men's energy. Therefore, the name “Sagunja-tang” is also used in the name of this landscape architecture formation and flower planting formation. It is the combination of the organic herbs including the ginseng, Atractylodis Rhizoma, Glycyrrhizae Radix, and Poria cocos that restores the men's energy. This Sagunja-tang landscape architecture is therefore characterized by the plants, trees, flowers, etc. that are the prescribed ingredients of the herbal mixture of Sagunja-tang that helps revive the men's energy, which have been understood based on the clinically studied results. In the Sagunja-tang landscape architecture, Poria cocos is used as an ingredient, but since it is a node of a pine tree's root, it is substituted with a Pinus densiflora (pine tree).

### B. Samul-tang

Oriental Medicine Prescription: Samul-tang

1. Classification: prescription for women.
2. Main ingredients: Angelicae gigantis radix, Cnidium rhizoma, Paeoniae radix, and Rehmanniae radix preparata.
3. Symptoms treated by the prescription: Symptoms treated by Samul-tang include: weak marrow hematosis ability; anemia due to low supply of iron content; dizziness; weakness; cold hands and feet; habitual miscarriages; blood discharge due to weak blood circulation. Samul-tang provides iron, strengthens the production of red blood cell by stimulating marrow thus reforming the symptom of blood discharge, and also promotes the growth of the uterus and strengthens the elasticity of the uterus muscles thus preventing miscarriage.
4. General Effects

a. Angelicae gignatis radix and Rehmannieae radix preparata increase production of the blood to relieve anemia.

b. Angelicae gignatis radix and Paeoniae radix expand blood vessel, and Cnidium rhizomapromotes blood circulation by strengthening the blood vessel artery.

c. Angelicae gignatis prevents miscarriages.

The Samul-tang Landscape Architecture

This formation of landscape architecture utilizes the Samul-tang prescription of the Oriental medicine and the prescribed herbs' effects and benefits classified according to the symptoms. The name, “Samul-tang,” is also used in the name of the landscape architecture to create familiarity.
1. Main landscaping materials: Angelica gigas Nakai, Cnidium oficinale, Paeonia lactiflora Pall, and Rebmannia glutinosa Libosch.
2. Characteristics of each main landscaping material:

a. Angelica gigas Nakai contains essential oils (scent), element of calmness along with saccharose, nicotine acid, folic acid, etc.

b. Cnidium officinale has roots that contain essential oils and various acids.

c. Paeonia lactiflora Pallcontains alkaloid, tannin, resin, sucrose, starch, salicylic acid, methyl salicylate, essential oils, etc.

d. Rehmannia glutinosa Libosch contains sucrose, carotin, cytostarol, vitamin A, catalpol, etc.
3. The medicinal effects of each landscaping material:

a. Angelica gigas Nakai provides a representative herbal treatment for nourishing blood and is used to cure anemia, menstrual problems, menstrual pains, and the lack of menstruation caused by bleeding during childbirth. It is used to cure heart beating, forgetfulness, insomnia, anxiety caused by blood discharging. It is good for healing bruises, stomach pain and hard excrement. It is also good for curing stomach aches, side aches, sore joints caused by clogged blood vessel and warms the body thus relieving labor pains.

b. Cnidium officinale is known to have the nature of high in heat, contains no poisonous elements, and spicy and bitter taste. It cleans the blood and is specially good for treating geriatric diseases such as the high blood pressure. It is also considered effective for treating obesity, pain, and gynecologic diseases.

c. Paeonia lactiflora Pall nourishes the blood, counterbalances one's virile powers, relieves muscle spasm, heals the stomach aches, and ease the hand and feet pains. Its primary element is a relaxer, and it is able to reduce pain and fever. It is good for patients with constipation problems since it relaxes the organs. Its peripheral effect is good for preventing ulcer.

d. Rehmannia glutinosa Libosch stops bleeding, reduces fever, strengthens heart, nourishes the blood, and helps to treat urination, diabetes, and the nervous breakdown.
4. The characteristics of the Samul-tang landscape architecture:

As an herbal medicine, Samul-tang is a type of restoration medicine to revive women's energy. Therefore, name “Samul-tang” is also used in the name of this landscape architecture formation and flower planting formation. It is a combination of organic herbs including Angelica gigas Nakai, Cnidium oficinale, Paeonia lactiflora Pall, and Rehmannia glutinosa Libosch to restore the women's energy. This Samul-tang landscape architecture is therefore characterized by the plants, trees, flowers, etc. that are prescribed ingredients of the herbal mixtures of Samul-tang, which helps revive the women's energy, based on the clinically tested results. In the Samul-tang landscape architecture, Rehmanniae radix preparata (which is a prescription ingredient and is a distillation of foxglove 9 times) is substituted with Rehmannia glutinosa Libosch.

### C. Palmul-tang

Oriental Medicine Prescription: Palmul-tang

1. Classification: a restorative herbal medicine.
2. Main ingredients: the ingredients of Sagunja-tang (ginseng, Atractylodis Rhizoma, Glycyrrhizae Radix, Poria cocos) plus the ingredients of the Samul-tang (Angelicae gigantis radix, Cnidium rhizoma, Paeoniae radix and Rehmanniae radix preparata).
3. Symptoms treated by the prescription: Palmul-tang controls the metabolism by strengthening the body capacity, strengthens the body immunity, increases the resistance against the stimulants to prevent aging, and promotes neutralizing ability in one's body.
4. General effects:

a. The ginseng, Atractylodis Rhizoma, Glycyrrhizae Radix, Poria cocos, Angelicae gigantis radix, Cnidium rhizoma, Paeoniae radix and Rehmanniae radix preparata nourish the body energy and blood.

b. Palmul-tang combines the invigorating effects of Sagunja-tang and the nourishing effects of Samul-tang so as to generally strengthen the whole body. It is effective for speedy recovery from a chronic exhaustion and treating all kinds of amenia and irregular miscarriage.

The Palmul-tang Landscape Architecture

This is a formation of landscape architecture utilizes the Palmul-tang prescription of the Oriental medicine and the prescribed herbs' effects and benefits classified according to the symptoms. The name, “Palmul-tang,” is also used in the name of the landscape architecture to create familiarity.
1. Main landscaping materials: the ginseng, Atractylodis Rhizoma, Glycyrrhizae Radix, Poria cocos, Angelicae gigantis radix, Cnidium rhizoma, Paeoniae radix and Rehmanniae radix preparata.
2. Characteristics of each main landscaping material: The Palmul-tang landscape architecture can be generally understood as the landscape architecture that combines the Sagunja-tang landscape architecture and the Samul-tang landscape architecture.
3. The medicinal effects of each landscaping material:

The Palmul-tang landscape architecture utilizes the materials of the Sagunja-tang and the Samul-tang landscape architectures as described above.
4. The characteristics of the Palmul-tang landscape architecture:

As an herbal medicine, Palmul-tang is of a type of medicine that restores and nourishes the entire human body. Therefore, the name “Palmul-tang” is also used in the name of this landscape architecture formation. It is the combination of invigorating Sagunja-tang and nourishing Samul-tang that generally strengthens the whole body. This Palmul-tang landscape architecture is therefore characterized by the plants, trees, flowers, etc. that are the prescribed ingredients of the herbal mixtures of Palmul-tang that helps nourish the entire body, based on the clinically tested results.

### D. Shipjundaebo-tang

Oriental Medicine Prescription: Shipjundaebo-tang

1. Classification: a restorative herbal medicine.
2. Main ingredients: the prescription ingredients of Palmul-tang and Astragalus root and Cinnamon branch.
3. Symptoms treated by the prescription: Shipjundaebo-tang is effective for treating anemia, loss of appetite, and the prolonged inflammation or infective wounds due to the declined immune system, weakened body and lack of strength (e.g., adynamia or asthenia) due to lack of cellular nutrients in the body cells. Shipjundaebo-tang strengthens the pivotal nerve abilities (central nervous system) by strengthening the body immune system. Shipjundaebo-tang stimulates the marrow to increased production of red blood cells in mass and promotes healthy blood circulation.
4. General effects:

a. Cinnamon branch, Angelicae gigantis radix, Cnidium rhizoma, Rehmanniae radix preparata, and paeoniae radix promote healthy blood circulation and reform anemia.

b. The ginseng, Atractylodis Rhizoma, and paconiac radix strengthen the gastric activities to improve digestion.

c. The ginseng, Poria cocos and Astragalus root provide nutrients to promote healthy metabolism and to prevent night sweating.

d. Astragalus root and Cinnamon branch heal ulcer.

e. Poria cocos and Atractylodis Rhizoma helps treat the swelling caused by weak blood vessels.

The “Shipjundaebo-tang” Landscape Architecture

This is a formation of landscape architecture utilizes the Shipjundaebo-tang prescription of the Oriental medicine and the prescribed herbs' effects and benefits classified according to the symptoms. The name, “Shipjundaebo-tang,” is also used in the name of the landscape architecture applied as it is to create familiarity.
1. Main landscaping materials: the ginseng, Atractylodis Rhizoma, Glycyrrhizae Radix, Poria cocos, Angelicac gigantis radix, Cnidium rhizoma, Paeoniae radix, Rehmanniae radix preparata, Astrualus membranaceus Bunge var, and Cinnamon.
2. Characteristics of each main landscaping material:

a. Astrualus membranaceus Bunge var contains glycoside, flavonoid, alkaloid, saccharine, amino acid, cholin, starch, etc.

b. Cinnamon—primary element is a type of aldehyde, also contains cinnamon liquid, starch, fluid, resin, etc.

c. The landscaping materials of the Palmul-tang landscape architecture.
3. The medicinal effects of each landscaping material:

a. Astrualus membranaceus Bunge var is a basic herb, which increases physical strength, eliminates anemia, manages sweating to make skin and muscle strong. As for women, Shipjundaebo-tang prevents bleeding from uterus mucous membrane and increases physical strength after or before a childbirth labor.

b. Cinnamon expands the blood vessels of coronary, strengthens the heart, changes the blood circulation and nutrition condition of skin, cures the chronic ulcer, stimulates sex hormones, protects liver, and prevents the reduction of glycogen. Cinamon effectively removes protein fat, increases immunity against various viruses such as grape diplococcus, pneumonia diplococcus, dysentery liver virus, diphtheria virus, etc.
4. The characteristics of the Shipjundaebo-tang landscape architecture:

The name “Shipjundaebo-tang” is also used in the name of this landscape architecture formation and flower planting formation. This Shipjundaebo-tang landscape architecture and flower planting are characterized by having the plants of the herbal mixtures of the Shipjundaebo-tang prescription, which help nourish the entire body, as this is based on the clinically tested results.

The following summarizes the benefits and the positive effects achieved by the various landscaping architectures disclosed in various embodiments of the present invention by applying the beneficial medicinal ingredients of plants, herbs, flowers, trees, etc. used for healing medicine in the Oriental medicine to the landscape architecture and flower planting:

1. The landscaping, gardening, flower planting, plant potting, etc. (collectively “the landscaping” or “the landscape”) techniques according to the present invention are distinctly different from the pre-existing and conventional landscape techniques. Thousands of the existing prescriptions of the Oriental medicine can be applied in addition to the prescriptions described above in creating the indoor and outdoor landscaping, flower planting, gardening, plant potting etc. according to the present invention. Further, they can be tailored according to each person's gender and physical constitutions, and accordingly various landscape architecture formations can be selected using the components and benefits of the herbs.

2. Numerous and diverse landscapes are possible according to the present invention by adding, deleting, and/or substituting the prescribed herbs and plants in the landscape. There are numerous oriental medicinal prescriptions and their names. The overall effect provided by the landscape formed according to the present invention depends on the addition and/or deletion of the plants, herbs, etc.

3. The landscape formed according to the present invention can substitute or replace the landscapes, gardens, etc. created by the conventional and existing techniques. The medicinal plants and herbs prescribed by the Oriental medicine includes many herbs and flowers having pleasant natural scent of their own, and they can substitute the ordinary flowers and plants in houses or offices.

4. The landscape according to the present invention fosters creation of a nature friendly environment. By directly cultivating and observing the growth of medicinal plants, flowers, etc. in their own landscape or garden, a more intimate and nature friendly environment can be created.

5. The landscaping techniques can be applied to exiting farmlands for cultivating relatively better income producing medicinal plants and herbs often prescribed by the Oriental medicine. For example, the landscape according to the present invention can be created on the rice or garlic producing farmlands (in Korea) that lost the economically competitive edge.

6. The landscape according to the present invention may serve a social role for the exchange of information between different occupational groups and/or between the East and the West. The exchange of information is achieved through the research of the medicinal plants and roots of the surrounding environment for development of the healing techniques that are environment and nature friendly as this applies to the landscape techniques of the present invention, and by applying this technique based on the Oriental medicine to other occupational groups in order to serve the social role of promoting the exchange of information.

Some of the specific examples of the application of the present invention are as the following:

1. The landscape customized according to one's physical constitution to promote his or her health as at least the aroma and fragrance of the plants in the landscape affects the person in a healthy way and provides therapeutic effects.

2. The landscape such as flower or plant pots, for example, customized as visiting gifts for patients in hospitals such that the flowers and/or the plants are selected according to the prescription of the Oriental medicine that may be used treat the patients' illnesses and symptoms.

3. The landscape such as flower or plant pots, for example, developed generally as healthy gifts.

4. The landscape according to the present invention generally utilized for landscaping the government facilities, public offices, buildings, streets, homes, etc.

5. The landscape according to the present invention providing the additional educational benefits of nature by raising herbs.

6. The herbs and plants grown by the landscaping techniques of the present invention being utilized for making tea or alcohol drinks.

1. A method of planting plants, together in close proximity to each other, that are substantially utilized as the medicinal ingredients in a selected single prescription of the traditional Oriental and Korean medicine wherein the plants provide therapeutic effects to the body of a person breathing the fragrance emitted by the plants and observing the plants.

2. The method of planting plants according to claim 1, wherein, in accordance with the prescription of Sagunja-tang, the plants are selected from the group consisting essentially of:
a ginseng;
a Atractylodes japonica koidz;
a Glycyrrhiza uralensis Fisch; and
a Pinus densiflora.

3. The method of planting plants according to claim 2, wherein the plants are planted in a portable medium including at least a flower pot.

4. The method of planting plants according to claim 1, wherein, in accordance with the prescription of Samul-tang, the plants are selected from the group consisting essentially of:
an Angelica gigas Nakia;
a Cnidium oficinale;
a Paeonia lactiflora Pall; and
a Rhemanniae glutinosa Libosch.

5. The method of planting plants according to claim 4, wherein the plants are planted in a portable medium including at least a flower pot.

6. The method of planting plants according to claim 1, wherein, in accordance with the prescription of Palmul-tang, the plants are selected from the group consisting essentially of:
a ginseng;
a Atractylodes japonica koidz;
a Glycyrrhiza uralensis Fisch;
a Pinus densiflora;
an Angelica gigas Nakia;
a Cnidium oficinale;
a Paeonia lactiflora Pall; and
a Rhemanniae glutinosa Libosch.

7. The method of planting plants according to claim 6, wherein the plants are planted in a portable medium including at least a flower pot.

8. The method of planting plants according to claim 1, wherein, in accordance with the prescription of Shipjundaebo-tang, the plants are selected from the group consisting essentially of:
a ginseng;
a Atractylodes japonica koidz;
a Glycyrrhiza uralensis Fisch;
a Pinus densiflora;
an Angelica gigas Nakia;
a Cnidium oficinale;
a Paconia lactiflora Pall;
a Rhemanniae glutinosa Libosch;
an Astrualus membranaceus Bunge var; and
a Cinnamon.

9. The method of planting plants according to claim 8, wherein the plants are planted in a portable medium including a flower pot.
